(19) United States
(12) Patent Application Publication
(10) Pub. No.: US 2005/0090896 A1
(43) Pub. Date: Apr. 28, 2005

(54) Intraocular lens

(76) Inventor: Yehoshua Ben Nun, Herzeliya (IL)

Correspondence Address: Nath & Associates, Sixth Floor, 1030 15th Street NW, Washington, DC, 20005, US

(21) Appl. No.: 10/468,465

(22) PCT Filed: Feb. 20, 2002
(86) PCT No.: PCT/IL02/00128

(30) Foreign Application Priority Data
Feb. 20, 2001 (IL) 141529

(51) Int. Cl. A61F 2/16

(52) U.S. Cl. 623/6.43; 623/6.38

(57) ABSTRACT

An intraocular lens system to be implanted in the posterior chamber of an eye, the system comprises a lens having an optical axis and at least two haptics extending from the circumference of the lens. The two haptics each includes one or more teeth located on their periphery.

### FIELD OF THE INVENTION

This invention relates to intraocular lens implantation and particularly to implantation in the posterior chamber of an eye.

### BACKGROUND OF THE INVENTION

Cataract is clouding of the natural lens of the eye or of its surrounding transparent membrane, which obstructs the passage of light causing various degrees of blindness. To correct this condition, a surgical procedure is known to be performed in which the opaque natural lens, or cataract, is extracted and replaced by an artificial intraocular lens.

The natural lens, located behind the iris in the posterior chamber in front of the vitreous cavity of the eye, is composed of a capsular bag containing gelatinous material. If this bag, called the posterior capsule, is left intact during a cataract extraction procedure, it may serve as a stable support site for implanting an intraocular lens. However, in the course of surgery, the posterior capsule may be inadvertently damaged or removed along with the cataract, in which case it would no longer be able to provide a support base to keep the intraocular lens from floating back into the vitreous cavity. In his case, it is known to implant the lens in the anterior chamber in front of the iris, or in the posterior chamber behind the iris, wherein the iris serves as a carrier for the lens in both instances. In the latter case, it has also been known to fix the intraocular lens in place behind the iris by suturing it to the ciliary sulcus. In both of the above cases, to maintain the intraocular lens properly centered, it is normally equipped with extensions, called haptics, which may have positioning holes to facilitate the centering of the lens.

U.S. Pat. No. 4,750,904 discloses a method of implanting an intraocular lens in the posterior chamber by tying the haptics to the iris and using small, radially disposed loops formed on the lens to serve as suture sites for securing the implanted lens directly to the iris.

### SUMMARY OF THE INVENTION

In accordance with the present invention, there is provided a novel solution for the self-fixation of an intraocular lens system in the posterior chamber of an eye. The lens system comprises a lens having an optical axis and at least two extending haptics attached to the circumference of the lens. These two haptics each have one or more teeth located on their periphery, which are particularly capable of penetrating the ciliary sulcus of the scleral wall of the eye, thereby anchoring the lens in place.

The teeth may be oriented to form an acute angle with the circumference of the haptic, thereby allowing free rotation of the haptic in one direction relative to the optical axis and allowing penetration of the teeth into the ciliary sulcus, when rotated in the other direction. The teeth may be harpoon-shaped, smooth or jagged in order to further facilitate their penetration or grasping of the ciliary sulcus.

The present invention provides for a secure self-attachment of the intraocular lens in the posterior chamber independently of the posterior capsule and without involving the iris.

### BRIEF DESCRIPTION OF THE DRAWINGS

In order to understand the invention and to see how it may be carried out in practice, different embodiments will now be described, by way of non-limiting examples only, with reference to the accompanying drawings, in which:

FIG. 1 is a schematic perspective view of an intraocular lens system according to the present invention;

FIG. 2 is a schematic view of the intraocular system shown in FIG. 1, when implanted in the posterior chamber of an eye;

FIGS. 3A, 3B, and 3C illustrate haptics of the lens system of FIG. 1, in accordance with three alternative embodiments of the present invention;

FIG. 4 is a schematic perspective view of an intraocular lens system according to another embodiment of the present invention;

FIG. 5 shows an optional supporting tool and a lens system according to the present invention, when supported by this tool for implantation;

FIG. 6 shows an optional protective sleeve for use with a lens system according to the present invention.

### DETAILED DESCRIPTION OF THE INVENTION

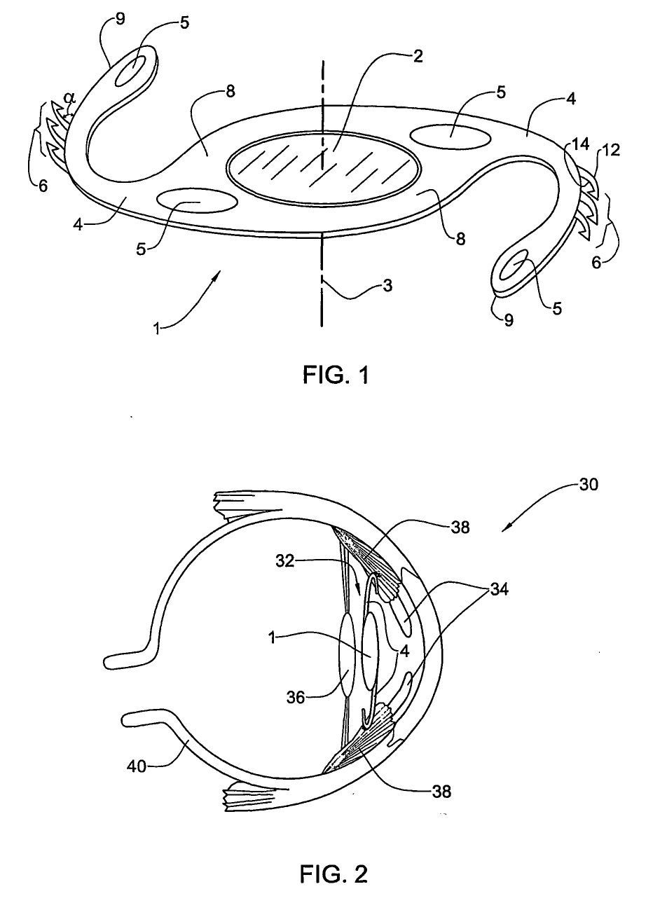

An intraocular lens system 1 in accordance with one embodiment of the present invention is shown in FIG. 1. The lens system 1 consists of a lens 2 of any known type having an optical axis 3 and two flexible haptics 4 extending away from the circumference of the lens 2. The haptics 4 include longitudinal positioning holes 5 near their base 8 and near their tip 9. These holes 5 aid in the positioning of the lens system 1 and facilitate manipulations thereof. The holes 5 may have any appropriate shape and each haptic 4 may have any number of them at any appropriate location.

With reference to FIG. 2, the lens system 1 is adapted to be implanted in an eye 30 to replace a cataract. FIG. 2 shows that, when implanted, the lens system 1 is located in the posterior chamber 32 between the iris 34 and the posterior capsule 36, with its haptics 4 bearing against the ciliary sulcus 38 of the scleral wall 40 of the eye 30.

In accordance with the present invention, and as seen in FIG. 1, each haptic 4 is provided with teeth 6 located at its periphery in the region designed to contact with the ciliary sulcus 38, when the lens system 1 is implanted in an eye. Thus, upon insertion of the lens system 1 into the eye 30 and manipulation of the haptics 4, the teeth 6 are made to penetrate and embed themselves in the ciliary sulcus 38 of the scleral wall 40, thereby securely anchoring the intraocular lens system 1 in the posterior chamber 32.

Reverting to FIG. 1, the teeth 6 may be harpoon-shaped with one smooth side 12 and one indented side 14, which is oriented to form an acute angle a with the circumference of the haptic 4. Such shape and orientation of the teeth 6 enable free rotation of the haptic 4 and the system 1 in one direction relative to the optical axis 3 and penetration and embedding of the teeth 6 into the ciliary sulcus 38, when rotation is attempted in the other direction.

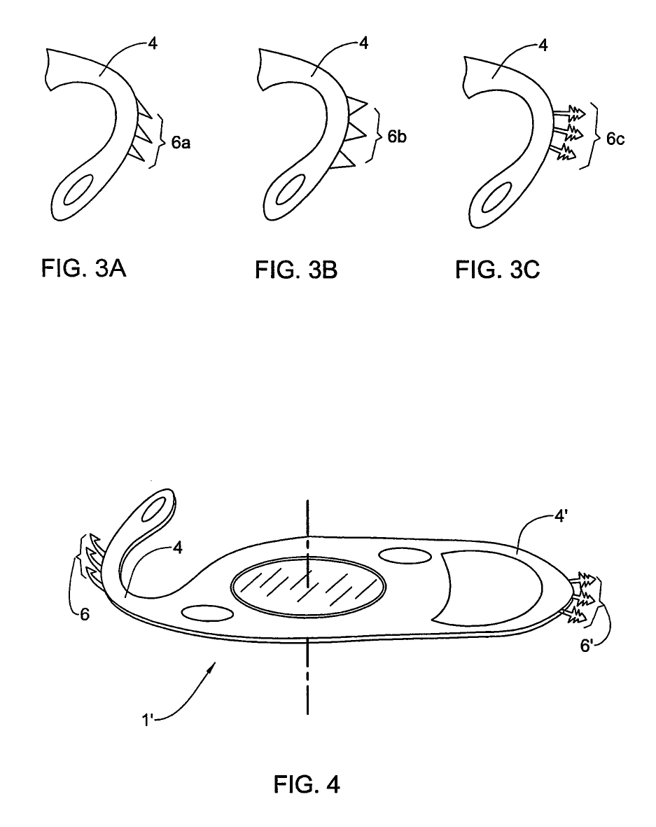

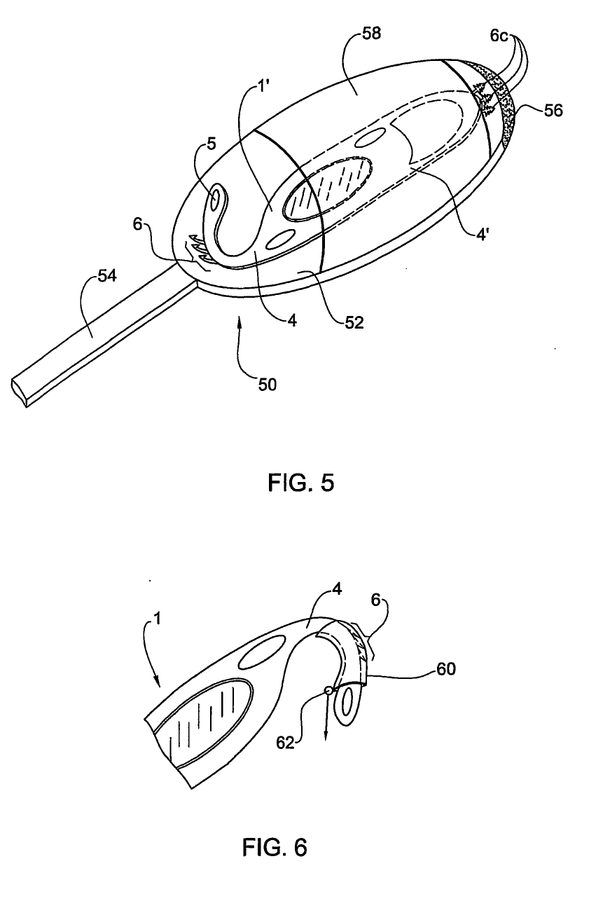

FIGS. 3A, 3B, and 3C show alternative designs for the teeth 6. In FIG. 3A, the teeth 6a are smooth on both sides and are acutely angled to allow rotation of the haptic 4 in only one direction. In FIG. 3B, the teeth 6b are similar to the teeth 6a but are oriented perpendicular to the circumference of the haptic 4 so as to prevent rotation in either direction. As shown in FIG. 3C, the teeth 6c are jagged to ensure an extremely firm anchoring of the haptics 4 in the eye 30.

To better secure the attachment of the lens system 1 and to center the lens 2, manipulation of the haptics 4 and the lens 2 can be performed by using the longitudinal positioning holes 5. It has been found that the longitudinal design of the positioning holes 5 is particularly useful as it allows for greater facility in the manipulation of the intraocular lens system 1 than conventional circular positioning holes. While oval positioning holes 5 are shown here, the longitudinal positioning holes 5 may be of various oblong shapes and sizes, with their length extending in the longitudinal direction of the haptic 4. Thus, rectangular or slit-like positioning holes may also be used. The oblong designs allow a surgeon to know when the intraocular lens system 1 has abutted the ciliary sulcus 38 during implantation.

In each haptic 4, 4′, any number of teeth 6 may be used and they may be placed anywhere on the far outer periphery of the haptics and at various distances from each other. The arrangement of the teeth on the two haptics of the lens system may be similar or completely different.

The lens 2 and haptics 4, 4′ may be produced as one body or alternatively, they may be produced as separate bodies, attachable to each other.

The teeth 6 may also be produced as one body along with the haptics 4, 4′ or rather they may be produced separately from the haptics, for example, to be attached thereto prior to implantation. In the latter case, it may be especially advantageous to produce a lens system to which teeth can be attached, when needed, and which can also be used without such teeth. For this purpose, the haptics should be formed with suitable teeth engagement means and/or the teeth may be formed with corresponding haptic engagement means.

The lens system of the present invention may be composed of various different substances. One example is making the teeth 6 of a biodegradable material, because it is beneficial to have the teeth 6 that penetrate the ciliary sulcus 38 completely dissolve over time. Another example is making the teeth 6 of a magnetic material so that they can be made to penetrate the sulcus 38 of the eye 30 by the use of an external magnet, after the haptic 4, 4′ has been positioned at the penetration site.

FIG. 5 illustrates an auxiliary tool 50 that may be used with an intraocular lens system of the present invention, for example, to facilitate its introduction into the eye 30. The auxiliary tool 50 may consist of a base 52 having a shape and dimensions to fully accommodate the lens system and a handle 54 attached to the base 52 at its end opposite its operative end 56. The tool 50 also has a covering member 58 to keep the lens system 1′ securely on the base 52 thereby restricting the motion of the lens system 1′ and reducing the likelihood of damaging the eye during implantation. While the auxiliary tool 50 should be rigid in order to direct the intraocular lens system 1′ into its operative position, its operative end 56 should preferably be soft and flexible to prevent damaging the eye. As an example of how this tool 50 can be used, an asymmetric intraocular lens system 1′ is shown thereon, having jagged teeth 6c on one haptic 4′ and harpoon-shaped teeth 6 on the other haptic 4. During its implantation, the lens system 1′ is brought to the ciliary sulcus 38 using the tool 50, and is pushed towards it, where the jagged teeth 6c are made to embed themselves firmly. The positioning holes 5 on the other haptic 4 are then used to embed the harpoon shaped teeth 6 at another desired site in the sulcus 38.

FIG. 6 shows an additional embodiment of the present invention, in which a thin protective sleeve 60 is placed over the teeth 6 so as to press them down to the haptic 4, thereby ensuring that they do not contact and damage the eye. The sleeve 60 should have a means for its removal from the haptic 4, such as an engaging loop 62, which can be pulled in the direction of the arrow to withdraw the sleeve 60 when the intraocular lens system 1 reaches the appropriate site of the ciliary sulcus 38, thereby exposing the teeth 6 and allowing them to penetrate the sulcus 38.

It should be understood that any permutation and/or combination of different features of the above-disclosed embodiments is also possible. It should further be understood that the above described embodiments constitute only examples of an intraocular lens system and a manner of its implantation according to the present invention, and that the scope of the present invention fully encompasses other embodiments which may become obvious to those skilled in the art.

1. An intraocular lens system to be implanted in the posterior chamber of an eye, the system comprising a lens having an optical axis and at least two haptics extending from the circumference of the lens, said two haptics each including one or more teeth located on their periphery.

2. An intraocular lens system according to claim 1, wherein said teeth are capable of penetrating the ciliary sulcus of the scleral wall of the eye, to anchor the lens in place.

3. An intraocular lens system according to claim 2, wherein at least one of said teeth is oriented to form an acute angle with the circumference of the haptic, so as to allow smooth rotation of the intraocular lens system in one direction relative to the optical axis and to enable penetration of the teeth into the ciliary sulcus when rotated in the other direction.

4. An intraocular lens system according to claim 1, wherein at least one of said teeth has a harpoon-shaped outer surface.

5. An intraocular lens system according to claim 1, wherein at least one of said teeth has a smooth outer surface.

6. An intraocular lens system according to claim 1, wherein at least one of said teeth has a jagged outer surface.

7. An intraocular lens system according to claim 1, wherein said lens and said haptics are produced as one body.

8. And intraocular lens system according to claim 1, wherein said haptics are produced as separate bodies, attachable to said lens.

9. An intraocular lens system according to claim 1, wherein said haptics and said teeth are produced as one body.

10. An intraocular lens system according to claim 1, wherein at least one of said teeth is produced as a separate body, attachable to at least one of said haptics.

11. An intraocular lens system according to claim 10, wherein said haptics are produced with teeth engaging means.

12. An intraocular lens system according to claim 10, wherein at least one of said teeth is made of a biodegradable material.

13. An intraocular lens system according to claim 10, wherein at least one of said teeth is made of a magnetic material.

14. An intraocular lens system according to claim 1, further including longitudinal positioning holes to facilitate the manipulation of the haptics and the lens.

15. An intraocular lens system according to claim 1, further including positioning holes at the haptic base and tip as to facilitate the manipulation of the haptics and the lens.

16. An intraocular lens system according to claim 1, further including a rigid tool to support the lens system for directing it into its operative position.

17. An intraocular lens system according to claim 16, wherein said tool is adapted to accommodate said lens system and to keep it secure by limiting its movement thereon.

18. An intraocular lens system according to claim 17, wherein said tool has an operative end, which is soft and flexible to avoid damaging the ciliary sulcus.

19. An intraocular lens system according to claim 1, further including a protective, possibly removable sleeve to be placed on at least one of said haptics for depressing said teeth thereon.

20. Haptic to be attached to an intraocular lens in a lens system defined in claim 1.

21. An intraocular lens system to be implanted in an eye, comprising a lens having an optical axis and at least two haptics extending from the periphery of the lens, said two haptics each being formed with teeth engaging means to enable the attachment thereto of one or more teeth.

22. An intraocular lens system according to claim 21, adapted to be implanted in the posterior chamber of an eye, wherein said engaging means is so located on the haptics as to enable the teeth, when attached thereto, to penetrate the ciliary sulcus of the scleral wall of the eye, to anchor the lens in place.
